(19) United States
(12) Patent Application Publication
(10) Pub. No.: US 2026/0009746 A1
(43) Pub. Date: Jan. 8, 2026

(54) METHOD FOR DETERMINING OIL SATURATION IN AN OIL LAYER

(71) Applicant: SCHLUMBERGER TECHNOLOGY CORPORATION, Sugar Land, TX (US)

(72) Inventors: Alexander Yuryevich DEMIANOV, Moscow (RU); Oleg Yurievich DINARIEV, Moscow (RU); Nikolay Vyacheslavovich EVSEEV, Moscow (RU); Evgeny Nikolaevich IVANOV, Moscow (RU)

(21) Appl. No.: 19/136,564

(22) PCT Filed: Dec. 4, 2023
(86) PCT No.: PCT/US2023/082345

§ 371 Date: Jun. 6, 2025

(30) Foreign Application Priority Data
Dec. 8, 2022 (RU) 2022132158

(51) Int. Cl. G01N 23/046; G01N 13/02; G01N 23/083; G01N 33/18; G01N 33/28

(52) U.S. Cl. CPC G01N 23/046; G01N 13/02; G01N 23/083; G01N 33/18; G01N 33/2823; G01N 2223/616

(57) ABSTRACT

The invention relates to methods for determining geological reserves of hydrocarbons, and particularly to methods for assessing the distribution of oil saturation in a reservoir. The technical result achieved by implementing the invention is to provide the ability to determine oil saturation and, accordingly, geological oil reserves with high accuracy by selecting sampling points for water, oil, and core sample and ensuring compliance with the geological conditions in the reservoir.

### CROSS-REFERENCE TO RELATED APPLICATIONS

The present application claims priority benefit of Russian Patent Application No. 2022132158, filed Dec. 8, 2022, the entirety of which is incorporated by reference herein and should be considered part of this specification.

### BRIEF DESCRIPTION OF THE DRAWINGS

FIG. 1 is a three-dimensional view of the distribution of water and oil in the pores of a digital model of a core sample at the end of drainage, emulating a laboratory technique.

FIG. 2 is a three-dimensional view of the distribution of water and oil in the pores of a digital model of a core sample for a case of global thermodynamic and mechanical equilibrium.

### DETAILED DESCRIPTION

The invention relates to methods for determining geological reserves of hydrocarbons, and in particular methods for assessing the distribution of oil saturation in a layer.

An important part of the effort of determining geological reserves of oil fields and development planning is assessing the distribution of oil saturation in a layer. Currently, this problem is solved using petrophysical correlations, e.g., saturation-permeability. These petrophysical correlations are obtained by processing results from laboratory analysis of two-phase (oil+water) saturations in core samples. There are several approaches for assessing saturation using laboratory methods. The first method is based on the ability to preserve the original fluid content in the core after the core has been extracted and transported to the surface. However, the core can potentially be contaminated by the drilling fluid, which generally reduces the reliability of this method. A more common approach is based on the following sequence of steps: a) purifying the core sample of any fluids present therein; b) saturating the core with water to a water saturation of 100%; and c) pumping oil into the water-saturated core until no more water is observed exiting the core. The fluid distribution in the core sample resulting from these operations is believed to represent the initial saturation of the layer. Processing a significant volume of results from such experiments can provide a basis for putting together the requisite petrophysical correlations, which can then be used to create a geological model of the layer (see, for example, Determination of Oil and Gas Reserves (Petroleum Society Monograph No.1) by The Petroleum Society of the Canadian Institute of Mining, Metallurgy and Petroleum, Calgary Section, 1994, pp. 35-105).

There are several problematic aspects inherent in the approach described above. First, there is no guarantee that laboratory techniques can obtain fluid saturations that correspond to the geological conditions in the reservoir. In fact, any laboratory experiment is going to be a great deal shorter than real geological processes of deposit formation. The distribution of fluids in pores obtained in a laboratory experiment may therefore differ from that which would be achieved in a geological process, since it may be possible to achieve a local thermodynamic equilibrium of fluids in laboratory time, but it will not necessarily approximate the global thermodynamic equilibrium achieved on a geological timescale.

Second, the properties of the fluids (water, oil) used in the experiment usually differ from the properties of real reservoir fluids, since model fluids are often used as experimental fluids. This adds uncertainty to the experimental results.

Finally, fluid saturation is often measured indirectly, such as by a correlation between saturation and electrical resistivity (the Archie-Dahnov correlation approach). This can be a source of additional errors.

The technical result achieved by implementing the present invention is to provide the ability to determine oil saturation and, accordingly, geological oil reserves with high accuracy by selecting sampling points for water, oil, and core samples, and ensuring correspondence to the geological conditions in the reservoir.

This technical result is achieved in accordance with the proposed method for determining oil saturation in an oil reservoir, by taking a deep sample of oil that is not contaminated with reservoir water or drilling fluid, and measuring the reservoir pressure at the sampling point.

Laboratory studies of the selected oil sample are carried out to determine the molar mass of oil, the dependence of the molar density of oil on reservoir pressure within a range characteristic of reservoir conditions, and the molar density of oil at reservoir pressure at the point of sampling the oil.

A deep sample of water that is not contaminated by reservoir oil or drilling fluid is taken, and the reservoir pressure at the sampling point is measured.

Laboratory studies of the selected oil sample are carried out to determine the molar mass of water, the dependence of the molar density of water on reservoir pressure within a range characteristic of reservoir conditions, and the molar density of water at reservoir pressure at the point of sampling the water.

The water-oil interfacial tension is determined.

At least one core sample is taken from the formation and X-ray computed microtomography of the selected sample is performed, and based on these results a three-dimensional digital model of the pore microstructure of the sample is constructed. The wettability of the sample pore walls is determined.

The Helmholtz free energy at unit volume of oil is determined using the obtained values of reservoir pressure at the oil sampling point, the molar mass of oil, the dependence of the molar density of oil on pressure within a range which is characteristic for reservoir conditions, and the molar density of oil at reservoir pressure at the oil sampling point.

The Helmholtz free energy at unit volume of oil is determined using the obtained values of reservoir pressure at the oil sampling point, the molar mass of oil, the dependence of the molar density of oil on pressure within a range which is characteristic for reservoir conditions, and the molar density of oil at reservoir pressure at the oil sampling point.

The Helmholtz free energy functional is constructed for the water-oil mixture in the pores using the obtained water-oil interfacial tension and wettability of the pore walls of the sample, the constructed three-dimensional model of the pore microstructure of the sample, and the calculated

Helmholtz energies per unit volume of water and oil, and the equilibrium distribution of water and oil is determined by minimizing the Helmholtz free energy functional.

The values of the chemical potentials of oil and water at the point of sampling the core sample are calculated, as is the distribution of oil and water in the pores, and the value of oil saturation at the point of sampling the core sample is determined.

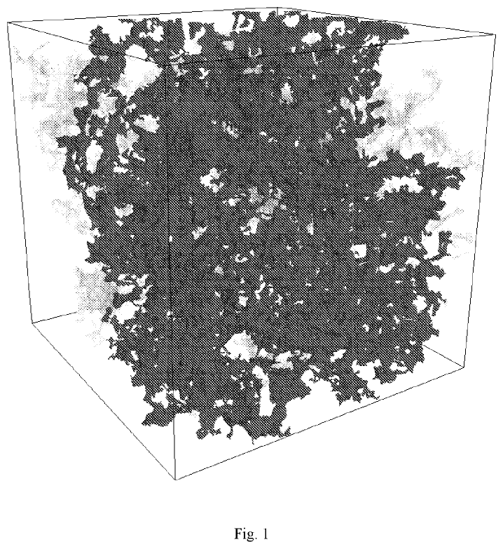

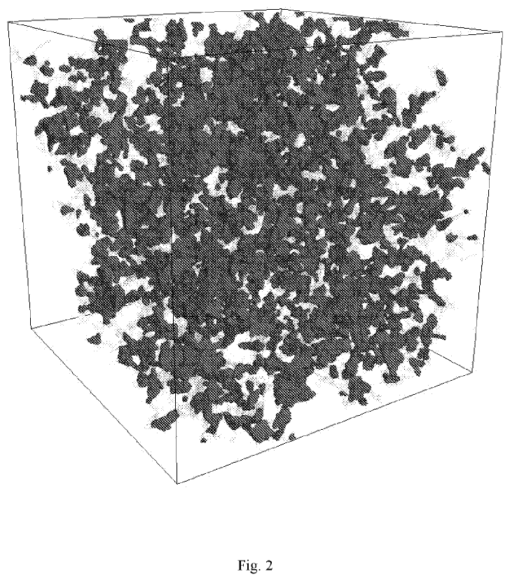

The present invention is illustrated by drawings. FIG. 1 shows a three-dimensional view of the distribution of water and oil in the pores of a digital model of a core sample at the end of drainage, emulating a laboratory technique, and FIG. 2 shows a three-dimensional view of the distribution of water and oil in the pores of a digital model core sample for the case of global thermodynamic and mechanical equilibrium.

In accordance with the proposed method, a deep oil sample is taken for laboratory research at a particular point A in the reservoir with an absolute depth mark. The sample must not be contaminated with reservoir water or drilling fluid. To avoid contamination, samples are preferably taken in the pure oil zone of the field before systematic development of the field begins, i.e., when the reservoir water is hydrodynamically motionless. Reservoir pressure at point A (pressure in the oil phase) will be denoted by P_{oilA}. This pressure can be measured using the Horner method, for example (Karnaukhov M. L., Pyankova E. M., Modern Methods of Hydrodynamic Well Testing. M.: Infra-Inzheneriya, 2010, pp. 69-75). The following parameters are determined by laboratory studies of the obtained oil sample (see, for example, Khaznaferov A. I., Study of Reservoir Oils. M.: Nedra, 1987, pp. 9-19, 46-69): molar mass of oil Mₒᵢₗ, dependence of molar density of oil nₒᵢₗ on pressure in oil pₒᵢₗ at reservoir temperature within the pressure range characteristic for reservoir conditions, and molar density of oil n_{oilA} at reservoir pressure p_{oilA}. A deep water sample is taken for laboratory research at a certain point B in the reservoir with an absolute depth mark h_{B}. The sample must not be contaminated with reservoir water or drilling fluid. Typically such a sample can be collected lower than where oil-water contact occurs. Reservoir pressure at point A (pressure in the oil phase) will be denoted by p_{watB}. This pressure can be measured using the Horner method, for example. The following parameters are obtained by laboratory measurements of a selected water sample: the molar mass of water M_{wat}, the dependence of the molar density of water n_{wat} on the pressure in water p_{wat}—within the pressure range characteristic for reservoir conditions, and the molar density of water n_{watB} at reservoir pressure p_{watB}.

The water-oil interfacial tension is determined using a known method (for example, Adamson A. W., Gast A. P., Physical Chemistry of Surfaces, New York: John Wiley and Sons, 1997, pp. 4-36).

A core sample is taken at a certain point C (or at a number of points) in the reservoir with an absolute depth mark h_{C} and examined using X-ray computed microtomography to construct a three-dimensional digital model of the pore microstructure of the sample (see, for example, Applications of X-ray Computed Tomography in the Geosciences, London: The Geological Society, 2003, pp. 23-60). Further, the wettability of the pore walls is determined using measurements or data on the mineralogy of the sample (Peters E. J., Petrophysics. Austin: University of Texas at Austin, 1997, pp. 6-31 to 6-45).

Point A is preferably selected so as to allow collecting a clean deep sample of oil (without possible contamination, for example, by reservoir water), and point B is preferably selected so as to allow collecting a clean deep sample of reservoir water. Point (or set of points) C is selected based on the need to obtain representative core samples for the oil reservoir under study.

Next, numerical modeling is done as follows.

The Helmholtz free energy at unit volume of oil is determined using the obtained values of reservoir pressure at point A, the molar mass of oil, the dependence of the molar density of oil p_{oilA} on pressure within a range which is characteristic for reservoir conditions, and the molar density of oil at reservoir pressure, and using the following equation. Mₒᵢₗ nₒᵢₗ pₒᵢₗ n_{oilA}.

$${f}_{oil} ( {n}_{oil} ) = {n}_{oil} {\int }_{{n}_{oil A}}^{{n}_{oil}} {n}^{- 2} {p}_{oil} ( n ) dn + ( {κ}_{oil A} - {n}_{oil A}^{- 1} {p}_{oil A} ) {n}_{oil} , \qquad ( 1 )$$

where κ_{oilA} is the chemical potential of the oil phase at point A, which is a constant that does not affect the final calculation results.

The Helmholtz free energy at unit volume of oil is determined using the obtained values of reservoir pressure at point B, the molar mass of oil, the dependence of the molar density of water p_{watB} on pressure within a range which is characteristic for reservoir conditions, and the molar density of water at reservoir pressure, and using the following equation. M_{wat} n_{wat} p_{wat} n_{watB} p_{watB}

$${f}_{wat} ( {n}_{wat} ) = {n}_{wat} {\int }_{{n}_{wat B}}^{{n}_{wat}} {n}^{- 2} {p}_{wat} ( n ) dn + ( {κ}_{wat B} - {n}_{wat B}^{- 1} {p}_{wat B} ) {n}_{wat} \qquad ( 2 )$$

where κ_{watB} is the chemical potential of the water phase at point B, which is a constant that does not affect the final calculation results.

Using the obtained water-oil interfacial tension, data on the wettability of the pore walls of the sample, the constructed digital three-dimensional model (or set of models) of the pore microstructure of the sample, as well as equations (1) and (2), a Helmholtz free energy functional F for the water-oil mixture in the pores is constructed using the technique described in A. Demianov, O. Dinariev and N. Evseev, Density functional modeling in multiphase compositional hydrodynamics, Can. J. Chem. Eng., 89, p. 206-226, 2011; A. Yu. Demyanov, O. Yu. Dinariev, N. V. Evseev, Fundamentals of the density functional method in hydrodynamics, Fizmatlit, 2009. For a given number of moles of oil and water Nₒᵢₗ, N_{wat} in the pore space, the equilibrium distribution of water and oil can be constructed by minimizing the Helmholtz free energy functional. The minimum value is a function of the variables Nₒᵢₗ, N_{wat}

$$F = F ( {N}_{oil} , {N}_{wat} ) \qquad ( 3 )$$

If variations in reservoir temperature are observed within the reservoir under consideration, then the dependence of the Helmholtz free energy functional on temperature is taken into account.

This function gives the chemical potential of the oil

$${κ}_{oil} = \frac{\partial {F}_{oil}}{\partial {N}_{oil}} \qquad ( 4 )$$

and water

$${κ}_{wat} = \frac{\partial {F}_{wat}}{\partial {N}_{wat}} \qquad ( 5 )$$

as functions of variables Nₒᵢₗ, N_{wat}.

The values of the chemical potentials of oil and water at point C are calculated under the condition of equilibrium in the reservoir

$${κ}_{oil C} = {κ}_{oil A} - {gM}_{wat} ( {h}_{C} - {h}_{A} ) , \qquad ( 6 )$$

$${κ}_{wat C} = {κ}_{wat B} - {gM}_{wat} ( {h}_{C} - {h}_{B} ) , \qquad ( 7 )$$

where g is the acceleration of gravity.

The following equations for variables Nₒᵢₗ, N_{wat} are solved using equations (4)-(7)

$${κ}_{oil} = {κ}_{oil C} \qquad ( 8 )$$

$${κ}_{wat} = {κ}_{wat C} \qquad ( 9 )$$

When obtaining the total number of moles of oil and water Nₒᵢₗ, N_{wat}, the distribution of oil and water in the pores and the oil and water saturation are simultaneously calculated. The distribution of oil and water in the pores is found as a state of the water-oil mixture, providing the minimum of the Helmholtz free energy functional. After that, the volume fraction of oil (oil saturation) and water (water saturation) is determined.

The procedure described above is carried out for a representative set of points C in order to determine the geological reserves of oil in the reservoir. After determining the amount of oil corresponding to the equilibrium geological conditions of occurrence in a representative set of points of the reservoir, the total reserves are calculated by summing over the volume of the reservoir after performing three-dimensional interpolation taking into account the geological structure of the object under consideration.

As an example of application of the described invention, the phase distribution of oil and water was calculated two ways: through displacement of water by pumping oil (emulating a laboratory approach) and through calculation of the equilibrium phase distribution. These calculations were performed on a digital model of a core sample taken from an oil field in Western Siberia.

A deep oil sample was taken in a pure oil zone from an exploration well at an absolute elevation of 1481 m, and the physical and chemical properties thereof were studied in accordance with standard methods (see, for example, Khaznaferov A. I., Study of Reservoir Oils. M.: Nedra, 1987, pp.9-19, 46-69).

A deep sample of reservoir water was taken in the aquifer zone from an exploration well at an absolute elevation of 1507 m, and the physical and chemical properties thereof were studied.

The core sample was extracted from the well at an absolute elevation of 1486 m. The hydrophilic type of rock wettability was determined. A three-dimensional model of the microstructure was constructed using X-ray microtomography.

Calculations of the initial oil saturation and residual water saturation were performed, with the following results (sₒᵢₗ, s_{wat}—oil and water saturation):
1) For the case of displacement of water by oil: sₒᵢₗ=0.59, s_{wat}=0.41 (see FIG. 1)
2) For the case of equilibrium phase distribution: sₒᵢₗ=0.68, s_{wat}=0.32 (FIG. 2)

Both calculations were carried out for the same fixed parameter h_{C}. In both cases, the chemical potentials of the oil and water were calculated using formulas (6) and (7).

Thereafter, in a first variation, the molar densities of the corresponding phases were determined in accordance with existing laboratory protocol, using expressions for the Helmholtz energies of oil and water (1) and (2). These molar densities were used in the numerical solution of the hydrodynamic problem of displacement of water by oil using the technique described in A. Demianov, O. Dinariev and N. Evseev, Density functional modeling in multiphase compositional hydrodynamics, Can. J. Chem. Eng., 89, pp. 206-226, 2011; A. Yu. Demyanov, O .Yu. Dinariev, N. V. Evseev, Fundamentals of the density functional method in hydrodynamics, Fizmatlit, 2009.

FIG. 1 is a three-dimensional view of the distribution of water and oil in the pores of a digital model of a core sample at the end of drainage, emulating laboratory technique (the water is shown in gray, and the oil in black).

In the second variation, the displacement problem was not attempted to be solved, but the equilibrium distribution of oil and water in the pores was calculated, corresponding to the known values of chemical potentials. This distribution was found as the conditional minimum point of the Helmholtz energy functional. FIG. 2 is a three-dimensional view of the distribution of water and oil in the pores of a digital model of a core sample for a case of global thermodynamic and mechanical equilibrium. Note that the detected increase in oil saturation corresponds to an increase in geological oil reserves by 15 percent.

Note that the values of the shear viscosity of oil and water were used to implement the calculation of the displacement of water by oil. However, fluid viscosities are not required to calculate the equilibrium distribution of fluids in the pore space, which is proposed in this invention.

1. A method for determining oil saturation in an oil reservoir, wherein:
a deep sample of oil that is not contaminated by reservoir water or drilling fluid is taken, and the reservoir pressure at the sampling point is measured,
laboratory studies of the selected oil sample are carried out to determine the molar mass of the oil, the dependence of the molar density of the oil on the reservoir pressure within a range characteristic of reservoir conditions, as well as the molar density of the oil at reservoir pressure at the oil sampling point,
a deep sample of water that is not contaminated by reservoir oil or by drilling fluid is taken, and the reservoir pressure at the sampling point is measured,
laboratory studies of the selected water sample are carried out to determine the molar mass of the water, the dependence of the molar density of the water on reservoir pressure within a range characteristic of reservoir conditions, and the molar density of the water at reservoir pressure at the water sampling point,
the water-oil interfacial surface tension is determined,
at least one core sample is taken from the reservoir and X-ray computed microtomography of the selected sample is performed, and based on these results, a three-dimensional digital model of the pore microstructure of the sample is constructed,
the wettability of the sample pore walls is determined,
the Helmholtz free energy per unit volume of the oil is determined using the obtained values of reservoir pressure at the oil sampling point, the molar mass of the oil, the dependence of the molar density of the oil on the pressure within a range characteristic of reservoir conditions, as well as the molar density of the oil at reservoir pressure at the oil sampling point,
the Helmholtz free energy at unit volume of oil is determined using the obtained values of reservoir pressure at the water sampling point, the molar mass of the water, the dependence of the molar density of the water on pressure within a range characteristic of reservoir conditions, and the molar density of the water at reservoir pressure at the water sampling point,
the Helmholtz free energy functional is constructed for the water-oil mixture in the pores using the obtained water-oil interfacial surface tension and wettability of the pore walls of the sample, the constructed three-dimensional model (or set of models) of the pore microstructure, the constructed three-dimensional model of the sample, and the calculated Helmholtz energies per unit volume of water and oil, and the equilibrium distribution of water and oil is determined by minimizing the Helmholtz free energy functional,
the values of the chemical potentials of oil and water at the core sample point are calculated, and the distribution of oil and water in the pores is calculated, and
the oil saturation value at the point of sampling the core sample is determined.
